(19) United States
(12) Patent Application Publication
(10) Pub. No.: US 2025/0239356 A1
(43) Pub. Date: Jul. 24, 2025

(54) METHODS AND SYSTEMS FOR ALLOCATING MEDICAL RESOURCES USING AN ARTIFICIAL INTELLIGENCE ENGINE

(71) Applicant: MedLever, Inc., Mountain View, CA (US)

(72) Inventors: Jacob S. PHILIP, Mountain View, CA (US); Loren Arnett VANDERBEEK, Palo Alto, CA (US); William WEILS, Sunnyvale, CA (US); Erwin NELL, Honolulu, HI (US)

(21) Appl. No.: 18/792,472

(22) Filed: Aug. 1, 2024

Related U.S. Application Data
(63) Continuation of application No. 18/605,424, filed on Mar. 14, 2024.
(60) Provisional application No. 63/624,231, filed on Jan. 23, 2024.

(51) Int. Cl. G16H 40/20

(52) U.S. Cl. CPC G16H 40/20

(57) ABSTRACT

Various methods, devices, and systems for task identification and resource management are described. In one aspect, a method includes obtaining information about a subject, obtaining identification of a condition of the subject based on the information, and obtaining identification of a treatment for the subject. The method also includes identifying a set of tasks corresponding to the treatment, generating, via a rules engine, a first set of parameters for the tasks, and generating, via an artificial intelligence (AI) engine, a second set of parameters for the tasks. The method further includes generating a resource allocation schedule for the treatment using the set of tasks, first set of parameters, and the second set of parameters, including assigning one or more tasks to a particular entity, and providing information about the one or more tasks to the particular entity.

### PRIORITY

This application is a continuation application to U.S. patent application Ser. No. 18/605,424, filed Mar. 14, 2024, which claims priority to U.S. Provisional Patent Application No. 63/624,231, filed Jan. 23, 2024, and titled “Methods and Systems for Allocating Medical Resources using an Artificial Intelligence Engine,” which is hereby incorporated by reference in its entirety.

### TECHNICAL FIELD

The disclosed embodiments relate generally to task identification and resource allocation including, but not limited to, systems and methods for using a combination of artificial intelligence and deterministic rules to generate resource allocation schedules and task assignments.

### BACKGROUND

Many professions require complex analysis where people need to consider many factors when identifying courses of action and allocating resources. For instance, a medical professional considering specific medical conditions, optimally should consider many different factors when assessing the patient's health as well as many factors when crafting and administering an optimized treatment plan. In addition, medical knowledge and treatment research are evolving rapidly so that medical professionals need to continually utilize data, new research, and new guidelines when selecting, scheduling, and/or updating treatment plans.

While conventional computers can provide data access and manipulation interfaces, they have several shortcomings. For example, conventional computers do not provide assistance with identifying conditions and/or treatment plans. Additionally, it can be cumbersome, and time consuming, to manually assign and schedule tasks using conventional interfaces.

### SUMMARY

The present disclosure describes, among other things, using artificial intelligence (AI) and/or machine learning to identify tasks and/or task parameters. For example, a medical professional may use an AI engine to identify an appropriate treatment for a patient (e.g., the AI engine may be configured to obtain and analyze medical information for the patient). In this example, the AI engine may identify available treatment options and present the options to the medical professional. After an appropriate treatment is identified, the AI engine may be used to identify a set of tasks corresponding to the treatment. The AI engine may obtain resource and/or scheduling information and schedule the set of tasks for particular times and with particular entities (e.g., personnel and/or equipment). In this way, the AI engine can provide a more efficient man-machine interface that requires less inputs from the user. Additionally, the AI engine can reduce user errors and mistakes and filter the data (e.g., patient data) to emphasize presentation of relevant data (and forgo presenting irrelevant or redundant data). In some circumstances the AI engine provides faster data processing and problem resolution than conventional data querying and analysis approaches.

In accordance with some embodiments, a method of task management includes (i) obtaining information about a subject via a user interface; (ii) identifying, based on the information about the subject, a set of tasks involving the subject, the set of tasks including a first subset of core tasks and a second subset of additional tasks; (iii) generating, via a rules engine, a first set of parameters for first subset of core tasks; (iv) generating, via an artificial intelligence (AI) engine, a second set of parameters for the second subset of additional tasks based on at least a subset of the information about the subject, at least a subset of the first set of parameters for the first subset of core tasks, and additional information obtained from one or more databases, wherein the first set of parameters and the second set of parameters each include one or more timing parameters and one or more ownership parameters; (v) generating a resource allocation schedule for the subject using the set of tasks, first set of parameters, and the second set of parameters, including assigning one or more tasks to a particular entity; and (vi) providing information about the one or more tasks to the particular entity.

In accordance with some embodiments, a method of resource allocation includes (i) obtaining information about a subject; (ii) obtaining identification of a condition of the subject based on the information about the subject; (iii) obtaining identification of a treatment for the subject based on the identification of the condition; (iv) identifying a set of tasks corresponding to the treatment; (v) generating, via a rules engine, a first set of parameters for the set of tasks; (vi) generating, via an artificial intelligence (AI) engine, a second set of parameters for the set of tasks based on at least a subset of the information about the subject, at least a subset of the first set of parameters, and additional information obtained from one or more databases; (vii) generating a resource allocation schedule for the treatment using the set of tasks, first set of parameters, and the second set of parameters, including assigning one or more tasks to a particular entity; and (viii) providing information about the one or more tasks to the particular entity.

In accordance with some embodiments, a computing system is provided. The computing system includes one or more processors (e.g., CPU(s), GPU(s), and/or NPU(s)) and memory storing one or more programs. The one or more programs include instructions for performing any of the methods described herein. In some embodiments, the computing system includes one or more user devices and/or one or more server systems.

In accordance with some embodiments, a non-transitory computer-readable storage medium is provided. The non-transitory computer-readable storage medium stores one or more programs for execution by a computing system with one or more processors. The one or more programs comprising instructions for performing any of the methods described herein.

Thus, devices and systems are disclosed with methods for task and resource allocation and management. Such methods, devices, and systems may complement or replace conventional methods, devices, and systems for task and resource allocation and management.

### BRIEF DESCRIPTION OF THE DRAWINGS

The embodiments disclosed herein are illustrated by way of example, and not by way of limitation, in the figures of the accompanying drawings. Like reference numerals refer to corresponding parts throughout the drawings and specification.

FIG. 1 is a block diagram illustrating an example system for task management and/or resource allocation in accordance with some embodiments.

FIG. 2 is a block diagram illustrating an example user device in accordance with some embodiments.

FIG. 3 is a block diagram illustrating an example server in accordance with some embodiments.

FIGS. 4A-4E illustrate an example user interface for patient and task management in accordance with some embodiments.

FIG. 5 is a flow diagram illustrating an example process of generating a resource allocation schedule in accordance with some embodiments.

FIG. 6 is a flow diagram illustrating an example process of updating a resource allocation schedule in accordance with some embodiments.

### DETAILED DESCRIPTION

The present application describes, among other things, systems, devices, and methods of task management and resource allocation using an AI engine. Using an AI engine to identify tasks, task parameters, and/or available resources improves the efficiency of man-machine interface (e.g., by reducing the number of user inputs) and improves user satisfaction (e.g., by presenting the user with content that they want to continue to engage with). As described in greater detail below, AI and machine-learning may be used to assist a medical professional in diagnosing a patient, identifying a suitable treatment plan for the patient based on the diagnosis, identifying tasks for the treatment plan, and/or identifying entities and timeslots for performing tasks. For example, an AI engine may be used to generate a resource allocation schedule, including automatically scheduling usage of one or more medical devices and/or automatically reserving time on respective calendars of one or more medical professionals.

Reference will now be made to embodiments, examples of which are illustrated in the accompanying drawings. In the following description, numerous specific details are set forth in order to provide an understanding of the various described embodiments. However, it will be apparent to one of ordinary skill in the art that the various described embodiments may be practiced without these specific details. In other instances, well-known methods, procedures, components, circuits, and networks have not been described in detail so as not to unnecessarily obscure aspects of the embodiments.

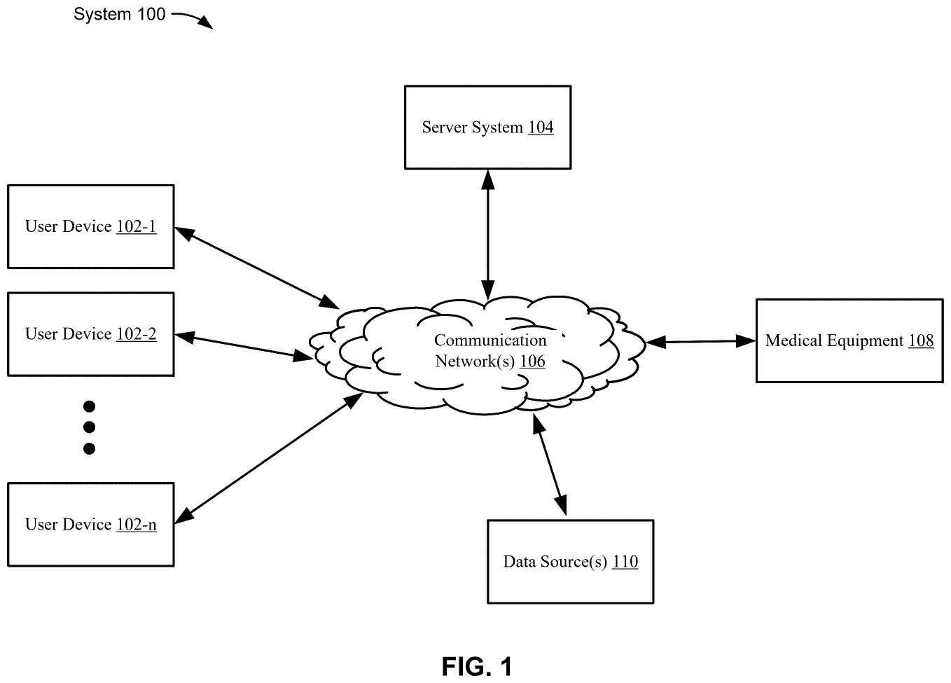

FIG. 1 is a block diagram illustrating a system 100 for task management and/or resource allocation in accordance with some embodiments. In some embodiments, the system 100 is configured to assist a medical professional in diagnosing a patient, identifying a treatment plan for the patient, identifying tasks for the treatment plan, and/or assigning appropriate entities and timeslots to the individual tasks.

The system 100 includes one or more user devices 102 (e.g., user device 102-1 through 102-n), a server system 104, one or more data sources 110, and medical equipment 108 (e.g., one or more medical devices). In some embodiments, the data sources 110 are a component of the server systems 104 and/or the user devices 102. For example, one or more of the data sources 110 may be stored in memory of the server system 104 and/or the user device(s) 102. The various components of the system 100 are communicatively coupled by one or more communication networks 106. In some embodiments, the one or more communication networks 106 include public communication networks, private communication networks, or a combination of both public and private communication networks. For example, the one or more communication networks 106 may be any network (or combination of networks) such as the Internet, other wide area networks (WAN), local area networks (LAN), virtual private networks (VPN), metropolitan area networks (MAN), peer-to-peer networks, and/or ad-hoc connections. In accordance with some embodiments, the system 100 includes, or communicates with, one or more external services and one or more external databases. In some embodiments, the system 100 includes only a subset of the components shown in FIG. 1. For example, in some implementations the system 100 may not include the data source(s) 110 and/or the medical equipment 108.

In some embodiments, a user device 102 is associated with one or more users. In some embodiments, a user device 102 is a personal computer, mobile electronic device, wearable computing device, laptop computer, tablet computer, mobile phone, feature phone, smart phone, an infotainment system, digital media player, a speaker, television (TV), and/or any other electronic device capable of receiving and responding to user inputs. The user devices 102 may connect to each other wirelessly and/or through a wired connection (e.g., directly through an interface, such as an HDMI interface). In some embodiments, the user devices 102-1 through 102-n are the same type of device. Alternatively, the user devices 102 may include two or more different types of devices. In some embodiments, the user device 102-1 communicates directly with another electronic device or computing system. For example, the user device 102-1 is able to communicate directly (e.g., through a wired connection and/or through a short-range wireless signal, such as those associated with personal-area-network (e.g., BLUETOOTH/BLE) communication technologies, radio-frequency-based near-field communication technologies, infrared communication technologies, etc.) with another device or computing system (e.g., the medical equipment 108, the data source(s) 110, and/or the server system 104).

In some embodiments, the user device(s) 102 send and receive information, such as queries and results, through the communication network(s) 106. For example, the user device(s) 102 may send a query or request to the server system 104, the data source(s) 110, and/or the medical equipment 108 through the communication network(s) 106. As another example, the user device(s) 102 may receive results and other responses from the server system 104, the data source(s) 110, and/or the medical equipment 108 through the communication network(s) 106. In some embodiments, two or more user devices 102 communicate with one another (e.g., resending and responding to queries and requests). The two or more user devices 102 may communicate via the communication network(s) 106 or directly (e.g., via a wired connection or through a peer-to-peer wireless connection).

In some embodiments, the server system 104 includes multiple electronic devices communicatively coupled to one another. In some embodiments, the multiple electronic devices are collocated (e.g., in a datacenter), while in other embodiments, the multiple electronic devices are geographically separated from one another. In some embodiments, the server system 104 stores and provides clinical and/or patient data. In some embodiments, the server system 104 trains, publishes, and/or utilities one or more engines and/or machine learning models. In some embodiments, the server system 104 receives and responds to queries and requests from the user device(s) 102 using the one or more engines and/or machine learning models. In some embodiments, the server system 104 includes multiple nodes and/or clusters configured to handle different types of tasks and/or handle requests and queries from different geographical locations.

In some embodiments, the user device(s) 102 and/or the server system 104 communicate with the data sources(s) 110 and/or the medical equipment 108 via respective application programming interfaces (APIs). In some embodiments, the data sources(s) 110 and/or the medical equipment 108 are maintained/operated by a third party to the system 100. In some embodiments, the system 100 includes, or is communicatively coupled to, one or more service(s) such as engines, assistants, location services, time services, web-enabled services, and/or services that access information stored external to the system 100.

As an example, a medical professional using the user device 102-1 may enter and/or retrieve patient information. For example, the patient information may be retrieved from the server system(s) 104, the data source(s) 110, the medical equipment 108, and/or an external database or service. The medical professional may provide the patient information to an AI engine executing at the user device 102-1 and/or the server system 104 and the AI engine may provide information about potential diagnoses, treatment plans, tasks, and the like.

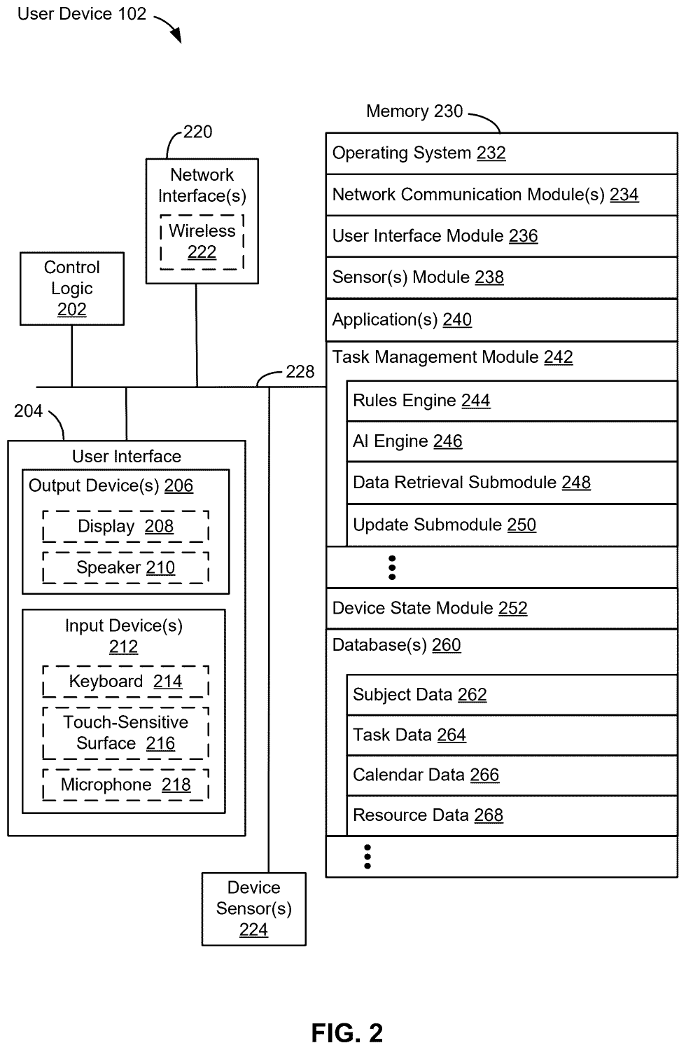

FIG. 2 is a block diagram illustrating a user device 102 in accordance with some embodiments. The user device 102 includes control logic 202, a user interface 204, one or more network interfaces 220, memory 230, and one or more communication buses 228 for interconnecting these components. The communication buses 228 optionally include circuitry (sometimes called a chipset) that interconnects and controls communications between system components. Optionally, the user device 102 includes a location-detection component, such as a global navigation satellite system (GNSS) (e.g., GPS (global positioning system), GLONASS, Galileo, BeiDou) or other geo-location receiver, and/or location-detection software for determining the location of the user device 102. In some embodiments, the control logic 202 includes one or more controllers, one or more CPUs, one or more GPUs, and/or one or more NPUs. In some embodiments, one or more CPU(s) are configured to perform operations related to an operating system 232, a network communications module 234, a user interface module 236, and/or a device state module 252. In some embodiments, the GPU(s) and/or the NPU(s) are configured operations related to an AI engine 246. In some embodiments, the network interface(s) 220 include one or more wireless interfaces 222 and/or one or more wired network interfaces. The user interface 204 includes one or more output device(s) 206 such as a display 208, a speaker 210, and/or other data output device. The user interface 204 also includes one or more input devices 212 such as a keyboard 214, a touch-sensitive surface 216, a microphone 218, and/or other user input device.

In some embodiments, user device 102 includes one or more device sensors 224 including, but not limited to, accelerometers, gyroscopes, compasses, magnetometer, light sensors, near field communication transceivers, barometers, humidity sensors, temperature sensors, proximity sensors, range finders, and/or other sensors/devices for sensing and measuring various environmental conditions.

In some embodiments, the one or more network interfaces 220 include wireless and/or wired interfaces for receiving data from and/or transmitting data to other user devices 102, the server system 104, and/or other devices or systems. The data communications may be carried out using any of a variety of custom or standard wireless protocols (e.g., NFC, RFID, IEEE 802.15.4, Wi-Fi, ZigBee, 6LoWPAN, Thread, Z-Wave, Bluetooth, ISA100.11a, WirelessHART, MiWi, etc.). Furthermore, the data communications may be carried out using any of a variety of custom or standard wired protocols (e.g., USB, Firewire, Ethernet, etc.). For example, the one or more network interfaces 220 may include a wireless interface 222 for enabling wireless data communications with other user devices 102, systems, and/or or other wireless (e.g., Bluetooth-compatible) devices. Furthermore, in some embodiments, the wireless interface 222 (or a different communications interface of the one or more network interfaces 220) enables data communications with other WLAN-compatible devices and/or the server system 104 (via the one or more communication network(s) 106).

The memory 230 includes high-speed random-access memory, such as DRAM, SRAM, DDR RAM, or other random access solid-state memory devices; and may include non-volatile memory, such as one or more magnetic disk storage devices, optical disk storage devices, flash memory devices, or other non-volatile solid-state storage devices. The memory 230 optionally includes one or more storage devices remotely located from the control logic 202. The memory 230, or, alternatively, the non-volatile solid-state memory device(s) within the memory 230, includes a non-transitory computer-readable storage medium. In some embodiments, the memory 230, or the non-transitory computer-readable storage medium of the memory 230, stores the following programs, modules and data structures, or a subset or superset thereof:
an operating system 232 that includes procedures for handling various basic system services and for performing hardware-dependent tasks;
a network communications module 234 that is used for connecting the user device 102 to other computing devices via the one or more network interfaces 220 (wired or wireless);
a user interface module 236 for processing inputs received via the user interface 204 and for generating outputs to be presented via the user interface 204;
a sensor(s) module 238 for receiving, processing, transmitting, and/or presenting information from the device sensor(s) 224;
one or more applications 240 for performing various functions with respect to providing and managing services, such as applications for word processing, calendaring, mapping, weather, stocks, time keeping, virtual digital assistant, presenting, number crunching (spreadsheets), drawing, instant messaging, e-mail, telephony, video conferencing, photo management, video management, a digital music player, a digital video player, 2D gaming, 3D (e.g., virtual reality) gaming, electronic book reader, and/or workout support;
a task management module 242 for identifying, configuring, and/or assigning tasks (e.g., for a treatment or procedure). In some embodiments, the task management module 242 is configured to allocate resources in accordance with entity scheduling and expertise information. In some embodiments, the task management module 242 includes one or more of:
   a rules engine 244 (e.g., a deterministic engine) for identifying tasks and/or task parameters (e.g., core and/or hardcoded tasks and parameters);
   an AI engine 246 for identifying tasks and/or task parameters, allocating resources, assigning entities to tasks, and/or updating tasks (e.g., based on data from one or more databases such as the data source(s) 110). In some embodiments, the AI engine 346 includes one or more language models, such as GPT-3, GPT-4, BioGPT, and PaLM-2;
   a data retrieval submodule 248 for retrieving data from one or more databases and/or other data sources (e.g., the data source(s) 110). In some embodiments, the data retrieval submodule 248 is configured to edit and/or process the data (e.g., vectorize or otherwise reformat the data); and
   an update submodule 250 for updating tasks and/or resource allocations (e.g., in conjunction with the rules engine 244 and/or the AI engine 246);
a device state module 252 for adjusting and/or maintaining a device state for the user device 102; and
one or more databases 260 for handling the storage of and/or access to data and/or metadata relating to the data. In some embodiments, the database(s) 260 are used by the one or more applications 240. In some embodiments, the database(s) 260 store data including:
   subject data 262 including subject data (e.g., patient data) such as medical information, subject details, and/or history information;
   task data 264 including data about current and past tasks (e.g., tasks identifying and/or assigned by the task management module 242);
   calendar data 266 including scheduling data for one or more users (e.g., medical professionals) and/or machines (e.g., medical devices); and
   resource data 268 including information about entities able and/or available to perform particular tasks.

Each of the above identified modules stored in the memory 230 corresponds to a set of instructions for performing a function described herein. The above identified modules or programs (e.g., sets of instructions) need not be implemented as separate software programs, procedures, or modules, and thus various subsets of these modules may be combined or otherwise re-arranged in various embodiments. For example, the task management module 242 does not include the update submodule 250 in some embodiments. In some embodiments, the memory 230 optionally stores a subset or superset of the respective modules and data structures identified above. For example, in some embodiments, the memory 230 includes a security module not shown in FIG. 2. The memory 230 optionally stores additional modules and data structures not described above. In some embodiments, the task management module 242 includes a natural language submodule (e.g., for parsing and/or generating natural language strings).

Although FIG. 2 illustrates the user device 102, in accordance with some embodiments FIG. 2 is intended more as a functional description of the various features that may be present in one or more media content servers than as a structural schematic of the embodiments described herein. In practice, and as recognized by those of ordinary skill in the art, items shown separately could be combined and some items could be separated. For example, some items shown separately in FIG. 2 could be implemented on single servers and single items could be implemented by one or more servers. In some embodiments, one or more of the database(s) 260 is stored on a device that is remote from the control logic 202. In some embodiments, at least a portion of the database(s) 260 is stored on a device that is accessed by the user device 102.

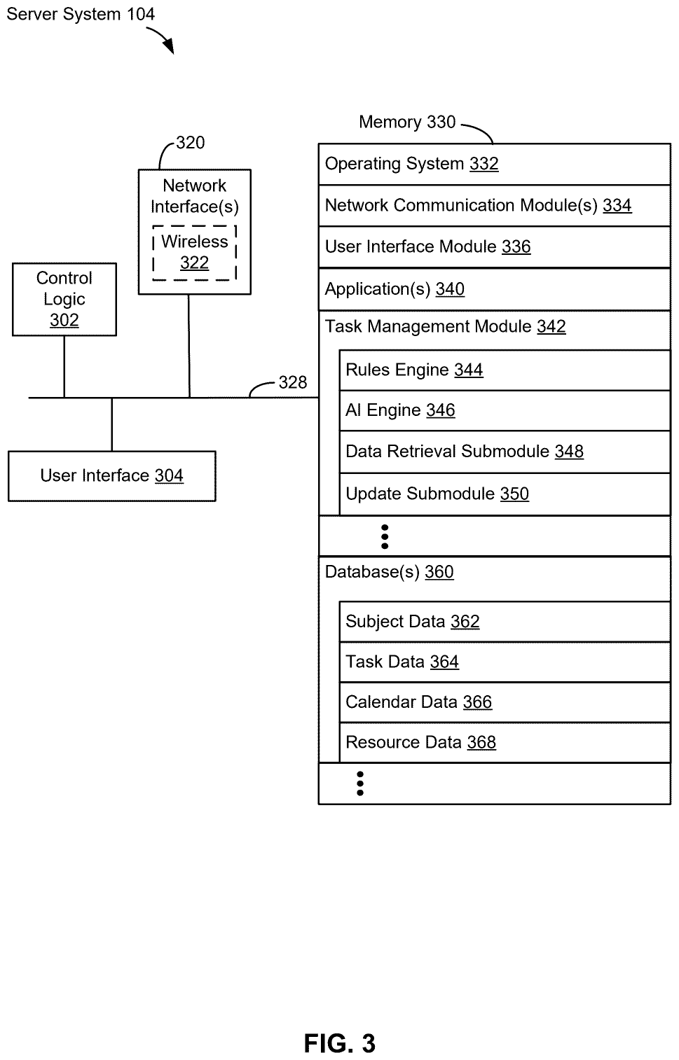

FIG. 3 is a block diagram illustrating an example server in accordance with some embodiments. The server system 104 includes control logic 302, a user interface 304, one or more network interfaces 320, memory 330, and one or more communication buses 328 for interconnecting these components. In some embodiments, the control logic 302 includes one or more controllers, one or more CPUs, one or more GPUs, and/or one or more NPUs. In some embodiments, the CPU(s) are configured to perform operations related to the operating system 332, the network communications module 334, the user interface module 336, and/or the applications 340. In some embodiments, the GPU(s) and/or the NPU(s) are configured to perform operations related to the task management module 342. In some embodiments, the user interface 304 includes a display and/or an input device or mechanism (e.g., a keyboard, a mouse, controller, and/or other type of input device). In some embodiments, the network interface(s) 320 include one or more wireless network interfaces 322 and/or one or more wired network interfaces.

The memory 330 includes high-speed random-access memory, such as DRAM, SRAM, DDR RAM, or other random access solid-state memory devices; and may include non-volatile memory, such as one or more magnetic disk storage devices, optical disk storage devices, flash memory devices, or other non-volatile solid-state storage devices. The memory 330 optionally includes one or more storage devices remotely located from the control logic 302. The memory 330, or, alternatively, the non-volatile solid-state memory device(s) within the memory 330, includes a non-transitory computer-readable storage medium. In some embodiments, the memory 330, or the non-transitory computer-readable storage medium of the memory 330, stores the following programs, modules and data structures, or a subset or superset thereof:
an operating system 332 that includes procedures for handling various basic system services and for performing hardware-dependent tasks;
a network communications module 334 that is used for connecting the server system 104 to other computing devices via the one or more network interfaces 320 (wired or wireless);
a user interface module 336 for processing inputs received via the user interface 304 and for generating outputs to be presented via the user interface 304;
one or more applications 340 for performing various functions with respect to providing and managing services;
a task management module 342 for identifying, configuring, and/or assigning tasks (e.g., for a treatment or procedure). In some embodiments, the task management module 342 is configured to allocate resources in accordance with entity scheduling and expertise information. In some embodiments, the task management module 342 operates in conjunction with the task management module 242 (e.g., the task management module 242 is a front end for the task management module 342). In some embodiments, the task management module 342 includes one or more of:
   a rules engine 344 (e.g., a deterministic engine) for identifying tasks and/or task parameters (e.g., core and/or hardcoded tasks and parameters);
   an AI engine 346 for identifying tasks and/or task parameters, allocating resources, assigning entities to tasks, and/or updating tasks (e.g., based on data from one or more databases such as the data source(s) 110). In some embodiments, the AI engine 346 includes one or more language models, such as GPT-3, GPT-4, BioGPT, and PaLM-2;
   a data retrieval submodule 348 for retrieving data from one or more databases and/or other data sources (e.g., the data source(s) 110). In some embodiments, the data retrieval submodule 348 is configured to edit and/or process the data (e.g., vectorize or otherwise reformat the data); and
   an update submodule 350 for updating tasks and/or resource allocations (e.g., in conjunction with the rules engine 344 and/or the AI engine 346); and
one or more databases 360 for handling the storage of and/or access to data and/or metadata relating to the data. In some embodiments, the database(s) 360 are used by the one or more applications 340. In some embodiments, the database(s) 360 store data including:
   subject data 362 including subject data (e.g., patient data) such as medical information, subject details, and/or history information;
   task data 364 including data about current and past tasks (e.g., tasks identifying and/or assigned by the task management module 242);
   calendar data 366 including scheduling data for one or more users (e.g., medical professionals) and/or machines (e.g., medical devices); and
   resource data 368 including information about entities able and/or available to perform particular tasks.

In some embodiments, the server system 104 includes one or more web or Hypertext Transfer Protocol (HTTP) servers, File Transfer Protocol (FTP) servers, as well as web pages and applications implemented using Common Gateway Interface (CGI) script, PHP Hyper-text Preprocessor (PHP), Active Server Pages (ASP), Hyper Text Markup Language (HTML), Extensible Markup Language (XML), Java, JavaScript, Asynchronous Javascript and XML (AJAX), XHP, Javelin, Wireless Universal Resource File (WURFL), and the like.

Each of the above identified modules stored in the memory 330 corresponds to a set of instructions for performing a function described herein. The above identified modules or programs (e.g., sets of instructions) need not be implemented as separate software programs, procedures, or modules, and thus various subsets of these modules may be combined or otherwise re-arranged in various embodiments. For example, the task management module 342 does not include the rules engine 344 in some embodiments. In some embodiments, the memory 330 optionally stores a subset or superset of the respective modules and data structures identified above. For example, in some embodiments, the memory 330 includes a security module not shown in FIG. 3. The memory 330 optionally stores additional modules and data structures not described above. In some embodiments, the task management module 342 includes a natural language submodule (e.g., for parsing and/or generating natural language strings).

Although FIG. 3 illustrates the server system 104, in accordance with some embodiments FIG. 3 is intended more as a functional description of the various features that may be present in one or more media content servers than as a structural schematic of the embodiments described herein. In practice, and as recognized by those of ordinary skill in the art, items shown separately could be combined and some items could be separated. For example, some items shown separately in FIG. 3 could be implemented on single servers and single items could be implemented by one or more servers. In some embodiments, the database(s) 360 are stored on devices that are remote from the control logic 302. In some embodiments, at least a portion of the database(s) 360 is stored on a device that is accessed by the server system 104. The actual number of servers used to implement the server system 104, and how features are allocated among them, will vary from one implementation to another and, optionally, depends in part on the amount of data traffic that the computing system handles during peak usage periods as well as during average usage periods.

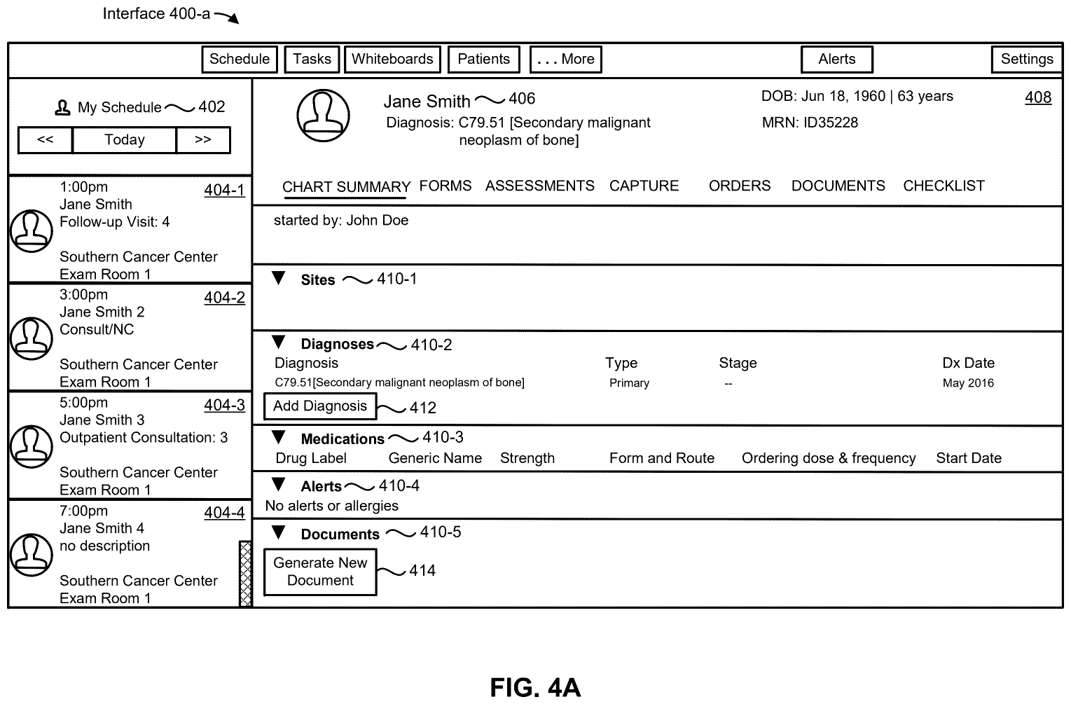

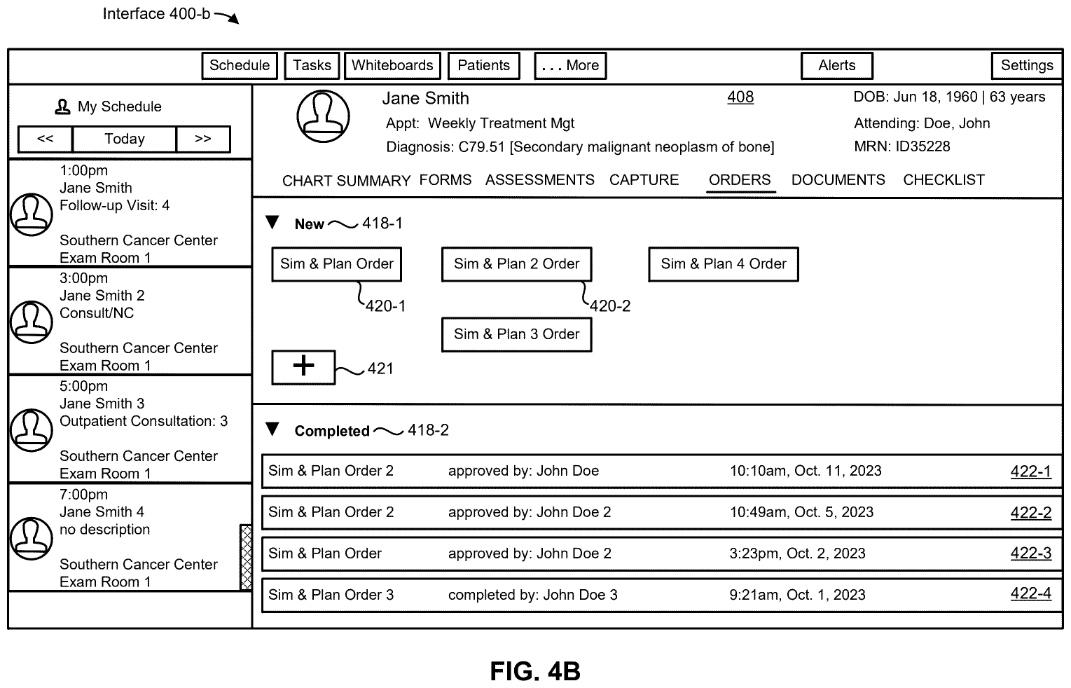

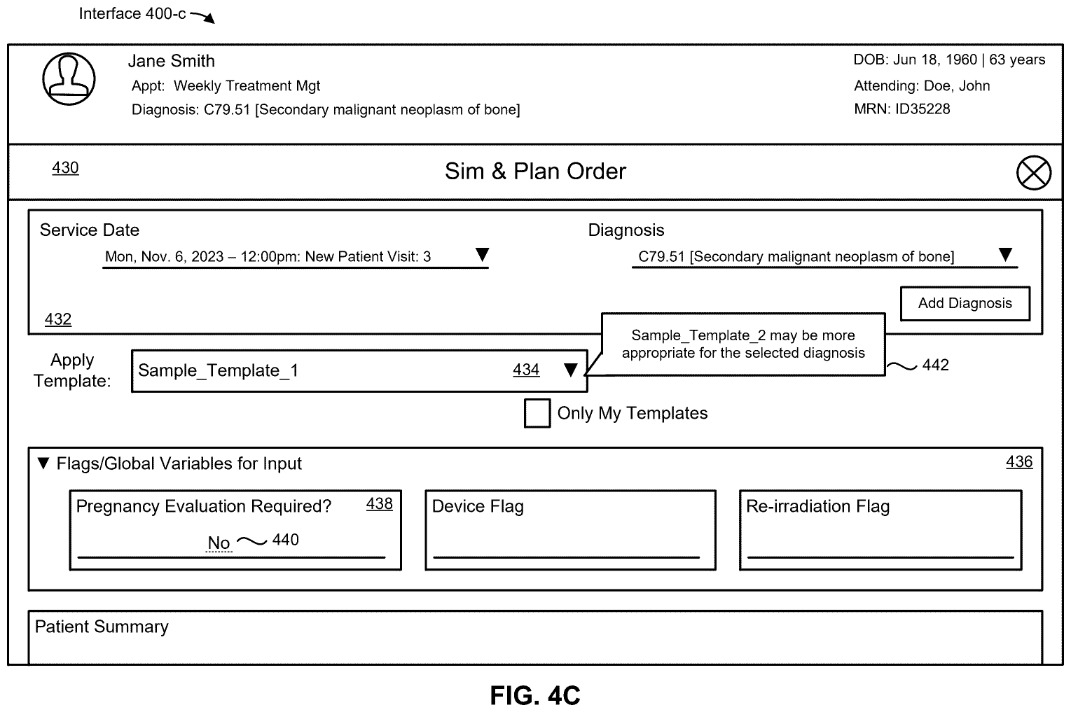

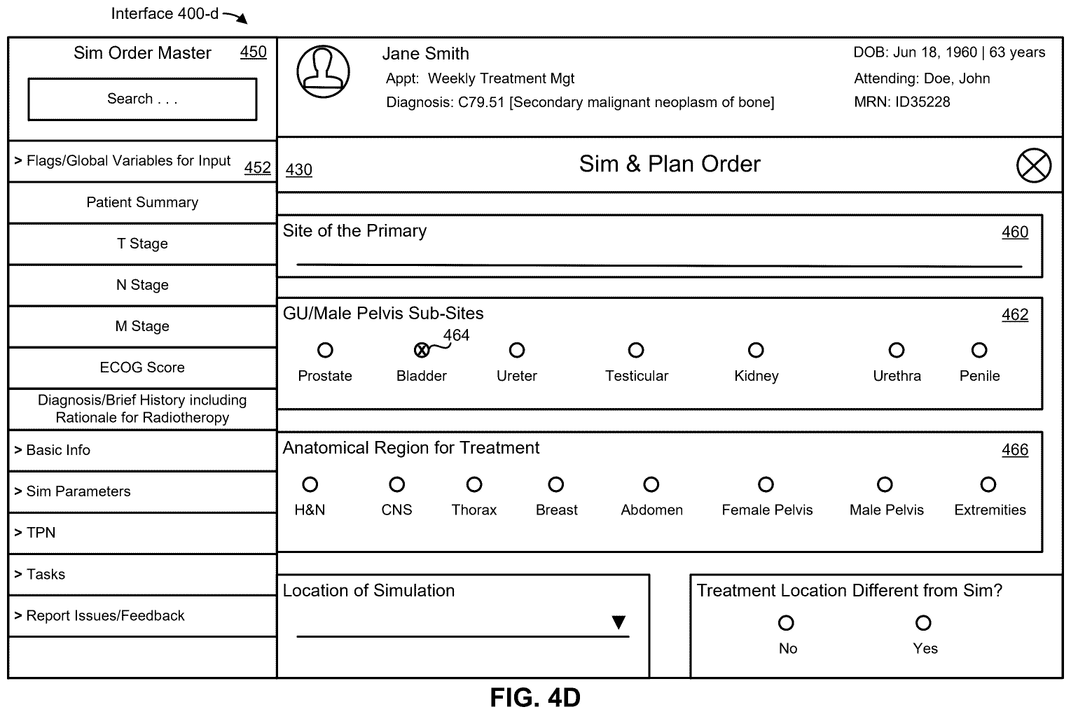

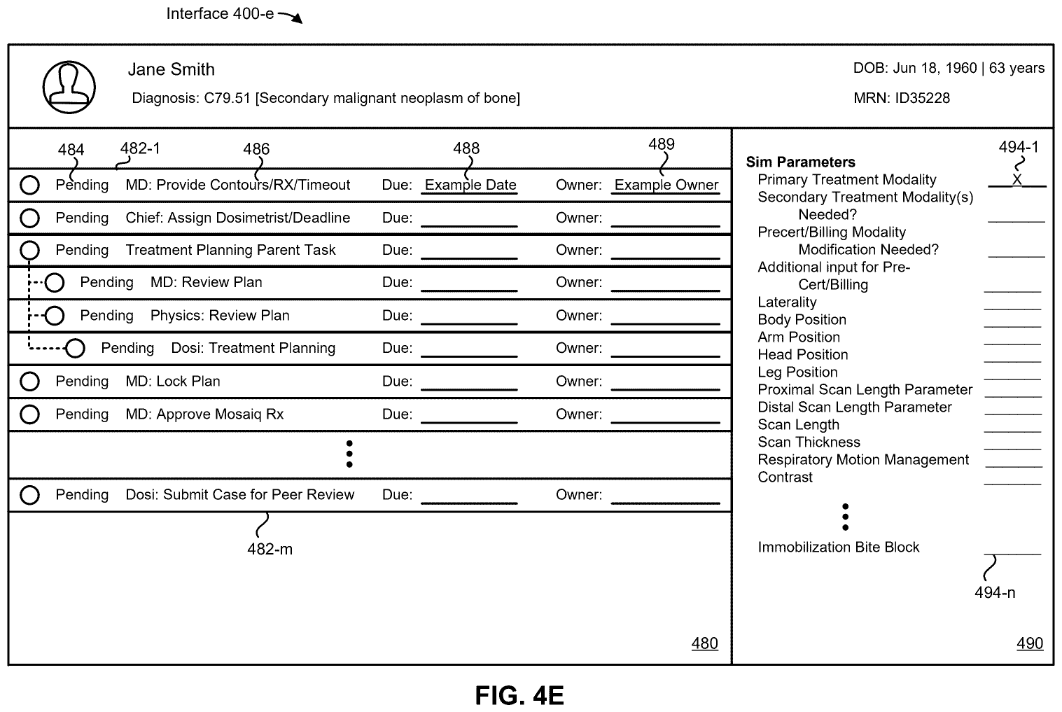

FIGS. 4A-4E illustrate an example user interface for patient and task management in accordance with some embodiments. FIG. 4A illustrates a user interface 400 in a state 400-a (e.g., a patient chart summary). The user interface 400 in FIG. 4A includes a section 408 for an example patient 406 (“Jane Smith”) that includes a chart summary. The chart summary has multiple sections 410 including a sites section 410-1 indicating active sites for the patient, a diagnoses section 410-2 indicating diagnoses for the patient, a medications section 410-3 indicating medications prescribed to the patient, an alerts section 410-4 that indicates any alerts or allergies of the patient, and a documents section 410-5 for viewing and/or editing documents associated with the patient. In accordance with some embodiments, the diagnoses section 410-2 includes an option 412 to add a diagnosis for the patient. In some embodiments, each of the sections 410 includes options to view, add, remove, and/or edit the information in the respective section.

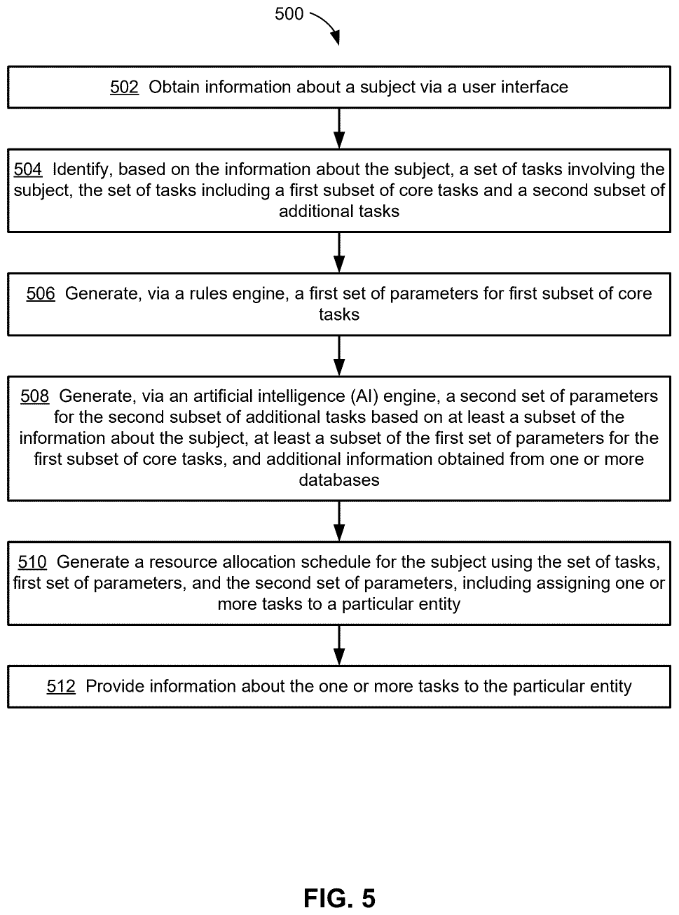

The user interface 400 in FIG. 4A includes a schedule section 402. The schedule section 402 includes information about a schedule of the user (e.g., a schedule for a medical professional). The schedule section 402 in FIG. 4A includes a listing of appointments 404 (e.g., appointment 404-1 through 404-4) with various patients. In some embodiments, the appointments 404 are generated in accordance with a resource allocation operation (e.g., via the task management module 242 and/or the task management module 342). For example, appointments may be generated in accordance with the method 500 described below with respect to FIG. 5.

In accordance with some embodiments, the documents section 410-5 includes an option 414 to generate a new document. In some embodiments, the option 414, when activated, causes display of a user interface to assist the user in generating a new document for the patient (e.g., a letter, a summary, a form, or other type of document). In some embodiments, in response to the user identifying a document type, a new document is generated for the user. In some embodiments, the new document is generated and/or populated using one or more engines (e.g., the rules engine 244 and/or the AI engine 246). In some embodiments, the new document is generated and/or populated automatically using data from one or more database(s) (e.g., the database(s) 260 and/or 360).

FIG. 4B illustrates the user interface 400 in a state 400-b (e.g., presenting a patient order section). In some embodiments, the user interface state 400-b in FIG. 4B corresponds to a transition from the user interface state 400-a in FIG. 4A (e.g., in response to a user selecting the orders tab in the user interface). The user interface 400 in FIG. 4B includes a new orders section 418-1 and a completed orders section 418-2. The new orders section 418-1 includes options 420 for generating new orders. For example, an option 420-1, when activated, initiates a new “Sim & Plan” example order and the option 420-2, when activated, initiates a new “Sim & Plan 2” example order. In accordance with some embodiments, new order types may be generated by selecting option 421. The completed orders section 418-2 includes a listing of completed orders 422, including information about each completed order.

FIG. 4C illustrates the user interface 400 in a state 400-c presenting a first portion of an order form 430. In some embodiments, the user interface state 400-c in FIG. 4C corresponds to a transition from the user interface state 400-b in FIG. 4B (e.g., in response to a user selecting the option 420-1). The order form 430 includes a section 432 for indicating a service date and corresponding diagnosis, an option 434 to select an order template, and a section 436 for selecting flags and/or variables for the order form. In some embodiments, a template is automatically selected for the form (e.g., the option 434 is pre-populated). In some embodiments, a default template is selected. In some embodiments, the option 434 is populated with a user selection. In some embodiments, a notification 442 is provided in accordance with a determination (e.g., by an AI engine) that another template may be more appropriate (e.g., based on the patient information such as the selected diagnosis). The notification 442 in FIG. 4C is merely an example and in other embodiments other types of notifications are provided. In some embodiments, one or more of the flags and/or variables are automatically populated (e.g., by an AI engine) based on patient information, such as input 440 for variable 438. In some embodiments, the automatically populated values are indicated to the user (e.g., using underlining as shown for input 440, highlighting, or other types of emphasis). In some embodiments, the order form 430 is populated using data retrieved from the database(s) 260 and/or 360.

FIG. 4D illustrates the user interface 400 in a state 400-d presenting a second portion of the order form 430. In some embodiments, the user interface state 400-d in FIG. 4D corresponds to a transition from the user interface state 400-c in FIG. 4C (e.g., in response to a user scrolling down in the order form 430). The user interface 400 in FIG. 4D includes an outline section 450 with a listing of sections for the order form 430. In some embodiments, the sections may be expanded or contracted in response to user inputs. In some embodiments, in response to a user selection of a particular section, the view of the order form 430 is adjusted to present the particular section. For example, a user selection of section 452 in the outline section 450 causes display of the section 436 in the order form 430. The order form 430 in FIG. 4D includes a primary site section 460, a sub-sites section 462, and a treatment region section 466. In some embodiments, one or more values for the sections are populated by an AI engine, such as selection of option 464 in the sub-sites section 462, based on data retrieved from the database(s) 260 and/or 360. In some embodiments, one or more values are suggested by an AI engine (e.g., based on data retrieved from the database(s) 260 and/or 360).

FIG. 4E illustrates the user interface 400 in a state 400-e presenting task information for the example patient 406 in a section 480. The task information in FIG. 4E includes a set of tasks 482 (e.g., task 482-1 through task 482-m). Each task includes a status 484, a description 486, a due date 488, and an owner 489. In some embodiments, one or more of the tasks includes a location, assigned equipment, priority, a scheduled performance date, and/or other parameters associated with the task. In some embodiments, one or more of the tasks are dependent on other tasks (e.g., a subset of the tasks must be completed in a particular order). For example, in FIG. 4E, the task “MD: Review Plan” is dependent on the task “Treatment Planning Parent Task.” In some embodiments, at least a subset of the tasks 482 are identified by a machine learning component (e.g., the AI engine 246 and/or the AI engine 346) based on patient data (e.g., data from the database(s) 260 and/or 360). In some embodiments, parameters for the tasks 482 are identified by a machine learning component (e.g., the AI engine 246 and/or the AI engine 346). For example, the machine learning component may identify a performance date/time and/or an owner for at least a subset of the tasks. In some embodiments, a first subset of tasks are designated as core tasks (e.g., by the rules engine 244 and/or 344) and parameters of the core tasks are identified by a user and/or a rules engine. In some embodiments, parameters are determined for core tasks and then parameters are determined for the remaining tasks based on the determined parameters for the core tasks. In some embodiments, parameters determined by a machine learning component are indicated to the user (e.g., with underlining, highlighting, or other emphasis) so that the user may update/confirm the parameters.

The user interface 400 in FIG. 4E includes a sim parameters section 490 indicating a set of sim (treatment) parameters 494 corresponding to the set of tasks 482. In some embodiments, the sim parameters 494 are adjustable by a user. In some embodiments, adjusting the sim parameters 494 causes the set of tasks 482 to update (e.g., tasks are added and/or removed based on the sim parameters 494). In some embodiments, adjusting the sim parameters 494 causes parameters of the tasks 482 to update.

FIG. 5 is a flow diagram illustrating a method 500 of generating a resource allocation schedule in accordance with some embodiments. The method 500 may be performed at a computing system (e.g., including one or more servers and/or one or more user devices) having one or more processors and memory storing instructions for execution by the one or more processors. In some embodiments, the method 500 is performed by the system 100. In some embodiments, the method 500 is performed by executing instructions stored in the memory (e.g., the memory 230 and/or the memory 330) of the computing system.

The system obtains (502) information about a subject (e.g., the subject data 262 and/or 362) via a user interface (e.g., the interface 400). In some embodiments, the subject is a patient. In some embodiments, the information about the subject is obtained using the user interface module 236. In some embodiments, the system further obtains information about the subject from one or more database (e.g., the data sources 110). In some embodiments, the information about the subject includes medical information.

The system identifies (504), based on the information about the subject, a set of tasks involving the subject, the set of tasks including a first subset of core tasks and a second subset of additional tasks. In some embodiments, the set of tasks correspond to a treatment plan for the subject. In some embodiments, the set of tasks are identified using a task management application (e.g., the task management module 242 and/or 342).

The system generates (506), via a rules engine (e.g., the rules engine 244 and/or 344), a first set of parameters for first subset of core tasks. In some embodiments, the first set of parameters include task prioritization, key dates/times for the core tasks, key entities and/or equipment for the core tasks. In some embodiments, the core tasks are identified by a user of the system (e.g., a medical professional). In some embodiments, the first set of parameters are identified by the user of the system. For example, certain tasks are identified as needing to be performed on certain dates and/or by particular entities (e.g., to ensure proper treatment) while other tasks have more flexibility in timing and/or execution.

The system generates (508), via an artificial intelligence (AI) engine (e.g., the AI engine 246 and/or 346), a second set of parameters for the second subset of additional tasks based on at least a subset of the information about the subject, at least a subset of the first set of parameters for the first subset of core tasks, and additional information obtained from one or more databases. In some embodiments, the AI engine includes one or more machine learning models. In some embodiments, the AI engine includes a generative AI module. In some embodiments, the second set of parameters include one or more timing parameters, one or more entity assignments, and/or one or more task settings (e.g., indicating one of two or more ways a task could be executed).

The system generates (510) a resource allocation schedule (e.g., via the task management module 242) for the subject using the set of tasks, first set of parameters, and the second set of parameters, including assigning one or more tasks to a particular entity. In some embodiments, generating the resource allocation schedule includes assigning one or more tasks to particular medical professions and/or medical devices. In some embodiments, generating the resource allocation schedule includes adding appointment information to one or more entity calendars. In some embodiments, generating the resource allocation schedule includes notifying the subject of timing and/or location for one or more of the tasks.

The system provides (512) information about the one or more tasks to the particular entity (e.g., the information is provided via the user interface 400). In some embodiments, the information about the one or more tasks includes information about performance of the task(s) and/or scheduling information for the task(s). In some embodiments, the information is provided via an alert or notification. In some embodiments, the information is added to a scheduling application used by the particular entity. In some embodiments, the information is provided with an option to decline and/or an option to adjust timing or other parameters of the task(s). In some embodiments, the system is configured to reallocate resources in response to the particular entity selecting a decline or adjust option.

Although FIG. 5 illustrates a number of logical stages in a particular order, stages which are not order dependent may be reordered and other stages may be combined or broken out. Some reordering or other groupings not specifically mentioned will be apparent to those of ordinary skill in the art, so the ordering and groupings presented herein are not exhaustive. Moreover, it should be recognized that the stages could be implemented in hardware, firmware, software, or any combination thereof.

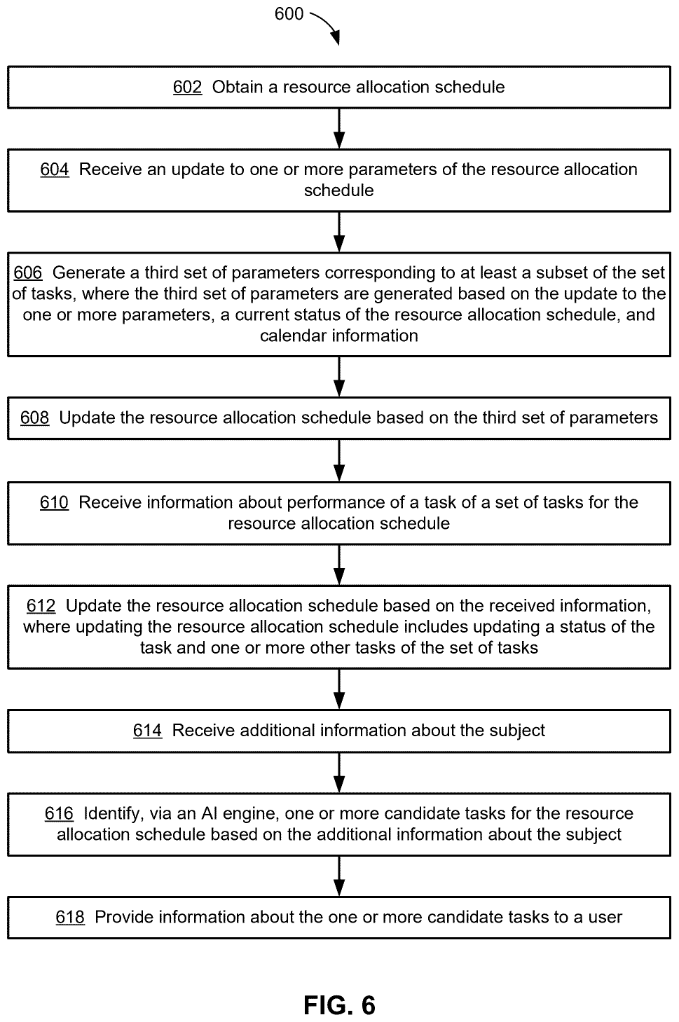

FIG. 6 is a flow diagram illustrating a method 600 of updating a resource allocation schedule in accordance with some embodiments. The method 600 may be performed at a computing system (e.g., including one or more servers and/or one or more user devices) having one or more processors and memory storing instructions for execution by the one or more processors. In some embodiments, the method 600 is performed by the system 100. In some embodiments, the method 600 is performed by executing instructions stored in the memory (e.g., the memory 230 and/or the memory 330) of the computing system. In some embodiments, the method 600 is performed subsequent to the method 500.

The system obtains (602) a resource allocation schedule (e.g., via the data retrieval submodule 348). In some embodiments, the resource allocation schedule is generated in accordance with the method 500 described previously. In some embodiments, the resource allocation schedule includes a set of tasks along with parameters for the set of tasks. In some embodiments, the resource allocation schedule is obtained from a database (e.g., one of the database(s) 260 and/or 360).

The system receives (604) an update to one or more parameters of the resource allocation schedule. For example, the update corresponds to a task owner (or assigned entity) declining, rescheduling, or otherwise revising the parameters of a particular task. In some embodiments, the updates are received via the user interface module 236 and/or the update submodule 350. In some embodiments, the update corresponds to a change in a treatment plan (e.g., due to test results). In some embodiments, the update corresponds to a change in availability of one or more resources.

The system generates (606) a third set of parameters corresponding to at least a subset of the set of tasks, where the third set of parameters are generated based on the update to the one or more parameters, a current status of the resource allocation schedule, and calendar information. In some embodiments, the third set of parameters are generated using a rules engine (e.g., the rules engine 244 and/or 344) and/or an AI engine (e.g., the AI engine 246 and/or 346). In some embodiments, the update to the one or more parameters is kept constant (e.g., hardcoded) and other parameters of the resource allocation schedule are recalculated accordingly to generate the third set of parameters.

The system updates (608) the resource allocation schedule based on the third set of parameters (e.g., via the update submodule 250 and/or 350). In some embodiments, the system identifies remaining tasks in the resource allocation schedule and reallocates resources for the remaining tasks based on the third set of parameters. In some embodiments, updating the resource allocation schedule includes indicating a priority for each remaining task.

The system receives (610) information about performance of a task of a set of tasks for the resource allocation schedule. For example, in response to a task status being updated (e.g., marked as completed), remaining task statuses are updated to indicate which tasks are ready to be performed next. In some embodiments, some tasks are identified as being dependent on other tasks. In these embodiments, the status of the dependent tasks are updated in response to the status of other tasks being updated. In some embodiments, a medical professional indicates that a task has been completed. In some embodiments, the system determines that a task has been completed (e.g., based on task data such as test results or other information generated during performance of the task).

The system updates (612) the resource allocation schedule based on the received information, where updating the resource allocation schedule includes updating a status of the task and one or more other tasks of the set of tasks. For example, the status of the task is set to completed or voided (e.g., determined to be unnecessary) and the status of a subsequent task is set to available to indicate that the subsequent task is ready to be performed.

The system receives (614) additional information about the subject (e.g., via the data retrieval submodule 248 and/or 348). In some embodiments, the additional information includes test results, health updates, and/or other subject information (e.g., availability and/or location updates). In some embodiments, the additional information is obtained via a user interface (e.g., the user interface 400). In some embodiments, the additional information is received from a medical professional. In some embodiments, the additional information is received from a database (e.g., the databases 260 and/or 360).

The system identifies (616), via an AI engine, one or more candidate tasks for the resource allocation schedule based on the additional information about the subject. For example, the system identifies potential tasks (e.g., additional tests and/or procedures) to include in the resource allocation schedule based on the additional information. For example, the additional information indicates that the subject has a particular response to a treatment and the candidate tasks correspond to revised treatment options based on the particular response.

The system provides (618) information about the one or more candidate tasks to a user (e.g., provides the information using the user interface module 236). For example, the information is provided via the user interface 400. In some embodiments, the information about the candidate tasks is provided to a medical profession (e.g., a doctor responsible for treating the subject). In some embodiments, in response to selection of one or more of the candidate task(s), the resource allocation schedule is updated to include the selected tasks.

Although FIG. 6 illustrates a number of logical stages in a particular order, stages which are not order dependent may be reordered and other stages may be combined or broken out. Some reordering or other groupings not specifically mentioned will be apparent to those of ordinary skill in the art, so the ordering and groupings presented herein are not exhaustive. Moreover, it should be recognized that the stages could be implemented in hardware, firmware, software, or any combination thereof.

Various example embodiments and aspects of the disclosure are described below for convenience. These are provided as examples, and do not limit the subject technology. Some of the examples described below are illustrated with respect to the figures disclosed herein simply for illustration purposes without limiting the scope of the subject technology.

(A1) In one aspect, some embodiments include a method of resource allocation (e.g., the method 500) that includes: (i) obtaining information about a subject (e.g., a patient); (ii) obtaining identification of a condition of the subject based on the information about the subject; (iii) obtaining identification of a treatment for the subject based on the identification of the condition; (iv) identifying a set of tasks corresponding to the treatment; (v) generating, via a rules engine (e.g., the rules engine 244 and/or 344), a first set of parameters for the set of tasks; (vi) generating, via an artificial intelligence (AI) engine (e.g., the AI engine 246 and/or 346), a second set of parameters for the set of tasks based on at least a subset of the information about the subject, at least a subset of the first set of parameters, and additional information obtained from one or more databases; (vii) generating a resource allocation schedule for the treatment using the set of tasks, first set of parameters, and the second set of parameters, including assigning one or more tasks to a particular entity; and (viii) providing information about the one or more tasks to the particular entity. In some embodiments, the information about the subject is obtained from one or more medical databases. In some embodiments, the identification of the treatment for the subject is obtained via the AI engine.

(B1) In one aspect, some embodiments include a method (e.g., the method 500) of task management that includes: (i) obtaining information about a subject via a user interface; (ii) identifying, based on the information about the subject, a set of tasks involving the subject, the set of tasks including a first subset of core tasks and a second subset of additional tasks; (iii) generating, via a rules engine, a first set of parameters for first subset of core tasks; (iv) generating, via an artificial intelligence (AI) engine, a second set of parameters for the second subset of additional tasks based on at least a subset of the information about the subject, at least a subset of the first set of parameters for the first subset of core tasks, and additional information obtained from one or more databases, wherein the first set of parameters and the second set of parameters each include one or more timing parameters and one or more ownership parameters; (v) generating a resource allocation schedule for the subject using the set of tasks, first set of parameters, and the second set of parameters, including assigning one or more tasks to a particular entity; and (vi) providing information about the one or more tasks to the particular entity. For example, the additional information may be obtained from a structured database (e.g., a SQL database) or from a cache (e.g., a redis cache). In some embodiments, generating the resource allocation schedule includes setting an initial status for each task of the set of tasks. In some embodiments, the rules engine identifies timing windows for the set of tasks and the AI engine identifies performance times for the set of tasks based on the timing windows. In some embodiments, the rules engine identifies potential owners for each task and the AI engine selects an owner from the potential owners (e.g., based on availability information). In some embodiments, the resource allocation schedule is a treatment resource allocation schedule. In some embodiments, the resource allocation schedule is a workflow. In some embodiments, generating the resource allocation schedule includes automatically scheduling usage of one or more medical devices and/or automatically reserving time on respective calendars of one or more medical professionals. In some embodiments, the rules engine is a deterministic rules engine.

(B2) In some embodiments of A1 or B1, the set of tasks are identified using the rules engine and/or the AI engine. In some embodiments, the set of tasks are customized and/or personalized to the subject, such as a personalized treatment plan (e.g., based on the information about the subject).

(B3) In some embodiments of any of A1, B1, or B2, the method further includes, prior to identifying the set of tasks: (i) identifying a set of options for the subject based on the information about the subject, where each option in the set of options has a corresponding set of tasks; and (ii) receiving a selection of a first option of the set options, wherein the set of tasks correspond to the first option. For example, based on a subject's medical condition different treatment options are identified. In this example, once a medical professional has selected one of the treatment options, a set of tasks is identified to carry out the selected treatment option. In some embodiments, the set of options is identified using the rules engine and/or the AI engine. In some embodiments, the AI engine includes a machine learning model trained to identify the set of options.

(B4) In some embodiments of any of A1 and B1-B3, the method further includes: (i) after generating the resource allocation schedule, receiving an update to one or more parameters of the resource allocation schedule; (ii) in response to the update, generating a third set of parameters corresponding to at least a subset of the set of tasks, where the third set of parameters are generated based on the update to the one or more parameters, a current status of the resource allocation schedule, and calendar information; and (iii) updating the resource allocation schedule based on the third set of parameters. For example, the update may be a manual update provided by a user or operator. As another example, the update may be generated based on an update to a different resource allocation schedule. For example, the update may be an availability of an entity assigned to a task (e.g., a medical professional has a schedule change, or a medical device requires maintenance). In some embodiments, the third set of parameters are generated by the rules engine and/or the AI engine.

(B5) In some embodiments of B4, updating the resource allocation schedule includes increasing a priority classification for one or more tasks of the resource allocation schedule. For example, one or more tasks are designated as having priority over tasks of other resource allocation schedules. In some embodiments, updating the resource allocation schedule includes rescheduling one or more tasks assigned to the particular entity (e.g., to prioritize more urgent tasks).

(B6) In some embodiments of any of A1 and B1-B5, the method further includes: (i) after generating the resource allocation schedule, receiving information about performance of a task of the set of tasks; and (ii) updating the resource allocation schedule based on the received information, where updating the resource allocation schedule includes updating a status of the task and one or more other tasks of the set of tasks. For example, the status of the task is set to completed and the status of a dependent task is changed from pending to ready to indicate that prerequisites for dependent task are now met. In some embodiments, the update is performed using the AI engine and/or the rules engine. In some embodiments, resource allocation schedules and/or individual tasks for other subjects and/or patients are also updated in accordance with updating the resource allocation schedule (e.g., to accommodate the change in availability of a task owner).

(B7) In some embodiments of B6, updating the resource allocation schedule comprises adjusting timing parameters for at least a subset of the set of tasks. For example, timing parameters may be adjusted based on information about the subject's health during performance of the task. In some embodiments, the method includes providing information about the adjusted timing parameters to a user and adjusting the timing parameters in accordance with approval from the user.

(B8) In some embodiments of any of A1 and B1-B7, the method further includes: (i) after generating the resource allocation schedule, receiving additional information about the subject; (ii) identifying, via the AI engine, one or more candidate tasks for the resource allocation schedule based on the additional information about the subject; and (iii) providing information about the one or more candidate tasks to a user. For example, the additional information may include toxicity information corresponding to a particular treatment. As another example, the additional information may include information about the subject's response to a particular therapy.

(B9) In some embodiments of B8, the method further includes, in response to a selection of the one or more candidate tasks by the user, generating a revised resource allocation schedule that incorporates the one or more candidate tasks. In some embodiments, generating the revised resource allocation schedule includes assigning a task to a second entity, and the method further comprises providing information about the task to the second entity.

(B10) In some embodiments of B8 or B9, the second set of parameters are generated by a first machine learning model of the AI engine, and the one or more candidate tasks are identified by a second machine learning model of the AI engine.

(B11) In some embodiments of any of A1 and B1-B10, the method further includes identifying, via the AI engine, one or more additional tasks for the resource allocation schedule, wherein the generated resource allocation schedule includes the one or more additional tasks. For example, the one or more additional tasks may be identified by machine learning model trained to identify resource allocation schedule tasks.

(B12) In some embodiments of any of A1 and B1-B11, the method further includes, in accordance with generating the resource allocation schedule, generating, via the AI engine, one or more documents corresponding to the resource allocation schedule based on the information about the subject. For example, the one or more documents may be generated by a machine learning model trained to generate documents. In some embodiments, the one or more documents are provided to a medical database (e.g., added to an electronic medical record for the subject). In some embodiments, the one or more documents are generated based on one or more tasks, one or more form parameters, one or more other documents, and/or additional information about the subject (e.g., information obtained from one or more databases).

(B13) In some embodiments of any of A1 and B1-B12, the set of tasks correspond to a treatment plan, therapy plan, and/or a medical procedure. For example, radiation oncology, surgery, cancer care, or other medical care.

(B14) In some embodiments of any of A1 and B1-B13, the method further includes assigning, via the AI engine, respective entities to the second subset of additional tasks, where the second set of parameters identify the respective assigned entities. For example, a machine learning model of the AI engine identifies the respective entities from a list of candidate entities based on an availability of each candidate entity.

(B15) In some embodiments of any of A1 and B1-B14, the AI engine includes a large language model. In some embodiments, the AI engine comprises one or more machine learning models (e.g., specialized models trained to perform different tasks).

(B16) In some embodiments of any of A1 and B1-B15, the AI engine is a generative artificial intelligence system. In some embodiments, the AI engine includes a natural language processing (NLP) component. For example, the NLP component may be configured to analyze unstructured text data and generate input vector(s) for other components of the AI engine. In some embodiments, the NLP component is configured to extract characteristics of the subject from unstructured data (e.g., to input to other components of the AI engine and/or to provide descriptive information, trends, and/or to prognosticate).

(B17) In some embodiments of any of A1 and B1-B16, the second set of parameters include: (i) a first subset of parameters, each parameter in the first subset of parameters identifying a respective owner assigned to a respective task of the second subset of additional tasks, and (ii) a second subset of parameters, each parameter of the second subset of parameters identifying a timing for performance of the respective task of the second subset of additional tasks. In some embodiments, the second set of parameters include task state parameters (e.g., indicating whether a current task state is pending, ready, in-progress, review required, completed, or not applicable).

(B18) In some embodiments of any of A1 and B1-B17, the at least the subset of the information about the subject, the at least the subset of the first set of parameters for the first subset of core tasks, and the additional information are provided to the AI engine as a vector of inputs. For example, log regression and/or deep learning is performed by the AI engine in response to receiving the vector of inputs (e.g., to identify top-k similarity results).

(B19) In some embodiments of any of A1 and B1-B18, the additional information comprises medical information and calendar information.

(B20) In some embodiments of B19, the medical information is obtained from one or more medical databases.

(B21) In some embodiments of B19 or B20, the medical information includes one or more of: a subject age, a subject gender, a health status of the subject, past treatment information for the subject, and a laboratory result for the subject. In some embodiments, the medical information includes diagnosis information, staging information, prior medical history for the subject, review of systems (RoS) information, and/or issues and complaints information. In some embodiments, the medical information includes diagnostics data such as text results, imaging studies, and/or other ‘omics data.

(B22) In some embodiments of any of B19-B21, the additional information further includes information about potential entities able to perform one or more tasks of the set of tasks, and wherein the calendar information comprises future calendar information for the potential entities.

(B23) In some embodiments of B22, the potential entities include one or more medical professionals and one or more medical devices.

(B24) In some embodiments of any of A1 and B1-B23, at least a subset of the set of tasks are dependent on one another. For example, a first task cannot be performed until a second task is completed.

(B25) In some embodiments of any of A1 and B1-B24, obtaining the information about the subject via the user interface comprises receiving the information from a user via a fillable electronic form. For example, the information corresponds to a diagnosis input by a medical professional.

(B26) In some embodiments of B25, the fillable electronic form is generated via a form builder based on data about the subject. For example, the data about the subject may comprise data from an electronic medical record of the subject and/or prior treatment information for the subject. In some embodiments, generating the fillable electronic form comprises pre-filling at least a portion of the form based on the data about the subject. In some embodiments, the fillable electronic form is generated by the AI engine (e.g., based on initial information about the subject and/or initial inputs from a medical professional). For example, the fillable electronic form may be progressively generated based on responses to earlier questions in the form.

(B27) In some embodiments of any of A1 and B1-B26, the method further includes training one or more models of the AI engine using a corpus of previously-performed resource allocation schedules. In some embodiments, the one or more models of the AI engine are trained using a corpus of medical information (e.g., electronic medical record (EMR) information) in addition to, or alternatively to, the information regarding previously-performed resource allocation schedules.

In another aspect, some embodiments include a computing system (e.g., the system 100) including control circuitry (e.g., the control logic 202 and/or the control logic 302) and memory (e.g., the memory 230 and/or 330) coupled to the control circuitry, the memory storing one or more sets of instructions configured to be executed by the control circuitry, the one or more sets of instructions including instructions for performing any of the methods described herein (e.g., the methods 500 and 600 as well as A1 and B1-B27 above).

In yet another aspect, some embodiments include a non-transitory computer-readable storage medium storing one or more sets of instructions for execution by control circuitry of a computing system, the one or more sets of instructions including instructions for performing any of the methods described herein (e.g., the methods 500 and 600 as well as A1 and B1-B27 above).

As used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1×10⁷. As such, the algorithms, models, regressors, and/or classifiers of the present disclosure cannot be mentally performed. In some embodiments n is between 10,000 and 1×10⁷, between 100,000 and 5×10⁶, or between 500,000 and 1×10⁶. In some embodiments, the algorithms, models, regressors, and/or classifier of the present disclosure operate in a k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

As used herein, the term “instruction” refers to an order given to a computer processor by a computer program. On a digital computer, each instruction is a sequence of 0s and 1s that describes a physical operation the computer is to perform. Such instructions can include data transfer instructions and data manipulation instructions. In some embodiments, each instruction is a type of instruction in an instruction set that is recognized by a particular processor type used to carry out the instructions. Examples of instruction sets include, but are not limited to, Reduced Instruction Set Computer (RISC), Complex Instruction Set Computer (CISC), Minimal instruction set computers (MISC), Very long instruction word (VLIW), Explicitly parallel instruction computing (EPIC), and One instruction set computer (OISC).

In some embodiments, the methods described herein include inputting information into a model comprising a plurality of parameters, wherein the model applies the plurality parameters to the information through a plurality of instructions to generate an output from the model.

In some embodiments, the plurality of parameters is at least 1000 parameters, at least 5000 parameters, at least 10,000 parameters is at least 50,000 parameters, at least 100,000 parameters, at least 250,000 parameters, at least 500,000 parameters, at least 1 million parameters, at least 5 million parameters, at least 10 million parameters, at least 25 million parameters, at least 50 million parameters, at least 100 million parameters, at least 250 million parameters, at least 500 million parameters, at least 1 billion parameters, or more parameters.

In some embodiments, the plurality of instructions is at least 1000 instructions, at least 5000 instructions, at least 10,000 instructions is at least 50,000 instructions, at least 100,000 instructions, at least 250,000 instructions, at least 500,000 instructions, at least 1 million instructions, at least 5 million instructions, at least 10 million instructions, at least 25 million instructions, at least 50 million instructions, at least 100 million instructions, at least 250 million instructions, at least 500 million instructions, at least 1 billion instructions, or more instructions.

It will also be understood that, although the terms first, second, etc. are, in some instances, used herein to describe various elements, these elements should not be limited by these terms. These terms are used only to distinguish one element from another. For example, a first electronic device could be termed a second electronic device, and, similarly, a second electronic device could be termed a first electronic device, without departing from the scope of the various described embodiments.

The terminology used in the description of the various embodiments described herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” is, optionally, construed to mean “when” or “upon” or “in response to determining” or “in response to detecting” or “in accordance with a determination that,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event]” or “in accordance with a determination that [a stated condition or event] is detected,” depending on the context.

The foregoing description, for purpose of explanation, has been described with reference to specific embodiments. However, the illustrative discussions above are not intended to be exhaustive or to limit the embodiments to the precise forms disclosed. Many modifications and variations are possible in view of the above teachings. The embodiments were chosen and described in order to best explain the principles and their practical applications, to thereby enable others skilled in the art to best utilize the embodiments and various embodiments with various modifications as are suited to the particular use contemplated.

1. A method of task management, comprising:
obtaining, from a first user, information about a subject via a user interface, the information comprising a diagnosis for the subject;
identifying, based on the information about the subject, a treatment plan corresponding to the diagnosis, the treatment plan comprising a set of tasks involving the subject, the set of tasks including a first subset of core tasks and a second subset of additional tasks;
generating, via a deterministic rules engine, a first set of parameters for the first subset of core tasks required by the treatment plan, wherein the first set of parameters indicates one or more timing windows for the set of tasks;
generating, via an artificial intelligence (AI) engine, a second set of parameters for the second subset of additional tasks based on at least a subset of the information about the subject, at least a subset of the first set of parameters for the first subset of core tasks, and additional information obtained from one or more databases, wherein the second set of parameters comprises one or more timing parameters corresponding to performance times within the one or more timing windows and one or more ownership parameters;
identifying, via the AI engine, one or more additional tasks related to the set of tasks;
generating a resource allocation schedule for the subject using the set of tasks, the one or more additional tasks, first set of parameters, and the second set of parameters, the generating including:
assigning a first set of one or more tasks of the resource allocation schedule to a first entity; and
assigning a second set of one or more tasks of the resource allocation schedule to a second entity;
causing generation of respective scheduling data for the first entity and the second entity, wherein the scheduling data corresponds to the performance times;
providing the respective scheduling data to the first and second entities, wherein providing the respective scheduling data comprises causing display of appointment information;
receiving, from a second user or system, a health update about the subject that causes an adjustment of priority of one or more tasks of the set of tasks;
in accordance with receiving the health update, generating an updated resource allocation schedule by updating one or more parameters of the resource allocation schedule according to the adjustment of priority, including generating updated respective scheduling data, wherein the updated respective scheduling data corresponds to different performances times than the respective scheduling data;
providing an indication of the updated resource allocation schedule to the first user; and
providing the updated respective scheduling data to the first and second entities.

2. The method of claim 1, wherein the set of tasks are identified using at least one of the deterministic rules engine and the AI engine.

3. The method of claim 1, further comprising, prior to identifying the set of tasks:
identifying a set of treatment options for the subject based on the information about the subject, wherein each treatment option in the set of treatment options has a corresponding set of tasks; and
receiving a selection of a first option of the set of treatment options, wherein the set of tasks correspond to the first option.

4. (canceled)

5. (canceled)

6. The method of claim 1, further comprising:
after generating the updated resource allocation schedule, receiving information about performance of a task of the set of tasks; and
further updating the resource allocation schedule based on the received information, wherein updating the resource allocation schedule includes updating a status of the task and one or more other tasks of the set of tasks.

7. The method of claim 6, wherein updating the resource allocation schedule comprises adjusting timing parameters for at least a subset of the set of tasks.

8. The method of claim 1, further comprising:
after generating the resource allocation schedule, receiving additional information about the subject, wherein the additional information comprises at least one of a test result for the subject, a health update for the subject, and availability of the subject;
identifying, via the AI engine, one or more candidate tasks for the resource allocation schedule based on the additional information about the subject; and
providing information about the one or more candidate tasks to the first user.

9. The method of claim 8, further comprising, in response to a selection of the one or more candidate tasks by the first user, generating a revised resource allocation schedule that incorporates the one or more candidate tasks.

10. The method of claim 8, wherein the second set of parameters are generated by a first machine learning model of the AI engine, and wherein the one or more candidate tasks are identified by a second machine learning model of the AI engine.

11. The method of claim 1, further comprising, in accordance with generating the resource allocation schedule, generating, via the AI engine, one or more documents corresponding to the resource allocation schedule based on the information about the subject.

12. (canceled)

13. The method of claim 1, further comprising assigning, via the AI engine, respective entities to the second subset of additional tasks, wherein the one or more ownership parameters identify the respective assigned entities, and wherein the respective assigned entities include the first entity and the second entity.

14. (canceled)

15. The method of claim 1, wherein the at least the subset of the information about the subject, the at least the subset of the first set of parameters for the first subset of core tasks, and the additional information are provided to the AI engine as a vector of inputs.

16. The method of claim 1, wherein the additional information comprises medical information and calendar information.

17. The method of claim 1, wherein the additional information comprises information about potential entities able to perform one or more tasks of the set of tasks and future calendar information for the potential entities.

18. (canceled)

19. A computing device, comprising:
one or more processors;
memory; and
one or more programs stored in the memory and configured for execution by the one or more processors, the one or more programs comprising instructions for:
obtaining, from a first user, information about a subject via a user interface, the information comprising a diagnosis for the subject;
identifying, based on the information about the subject, a treatment plan corresponding to the diagnosis, the treatment plan comprising a set of tasks involving the subject, the set of tasks including a first subset of core tasks and a second subset of additional tasks;
generating, via a deterministic rules engine, a first set of parameters for the first subset of core tasks required by the treatment plan, wherein the first set of parameters indicates one or more timing windows for the set of tasks;
generating, via an artificial intelligence (AI) engine, a second set of parameters for the second subset of additional tasks based on at least a subset of the information about the subject, at least a subset of the first set of parameters for the first subset of core tasks, and additional information obtained from one or more databases, wherein the second set of parameters comprises one or more timing parameters corresponding to performance times within the one or more timing windows and one or more ownership parameters;
identifying, via the AI engine, one or more additional tasks related to the set of tasks;
generating a resource allocation schedule for the subject using the set of tasks, the one or more additional tasks, first set of parameters, and the second set of parameters, the generating including:
assigning a first set of one or more tasks of the resource allocation schedule to a first entity; and
assigning a second set of one or more tasks of the resource allocation schedule to a second entity;
causing generation of respective scheduling data for the first entity and the second entity, wherein the scheduling data corresponds to the performance times;
providing the respective scheduling data to the first and second entities, wherein providing the respective scheduling data comprises causing display of appointment information;
receiving, from a second user or system, a health update about the subject that causes an adjustment of priority of one or more tasks of the set of tasks;
in accordance with receiving the health update, generating an updated resource allocation schedule by updating one or more parameters of the resource allocation schedule according to the adjustment of priority, including generating updated respective scheduling data, wherein the updated respective scheduling data corresponds to different performances times than the respective scheduling data;
providing an indication of the updated resource allocation schedule to the first user; and
providing the updated respective scheduling data to the first and second entities and to the first user.

20. A non-transitory computer-readable storage medium storing one or more programs configured for execution by a computing device having one or more processors and memory, the one or more programs comprising instructions for:
obtaining, from a first user, information about a subject via a user interface, the information comprising a diagnosis for the subject;
identifying, based on the information about the subject, a treatment plan corresponding to the diagnosis, the treatment plan comprising a set of tasks involving the subject, the set of tasks including a first subset of core tasks and a second subset of additional tasks;
generating, via a deterministic rules engine, a first set of parameters for the first subset of core tasks required by the treatment plan, wherein the first set of parameters indicates one or more timing windows for the set of tasks;
generating, via an artificial intelligence (AI) engine, a second set of parameters for the second subset of additional tasks based on at least a subset of the information about the subject, at least a subset of the first set of parameters for the first subset of core tasks, and additional information obtained from one or more databases, wherein the second set of parameters comprises one or more timing parameters corresponding to performance times within the one or more timing windows and one or more ownership parameters;
identifying, via the AI engine, one or more additional tasks related to the set of tasks;
generating a resource allocation schedule for the subject using the set of tasks, the one or more additional tasks, first set of parameters, and the second set of parameters, the generating including:
assigning a first set of one or more tasks of the resource allocation schedule to a first entity; and
assigning a second set of one or more tasks of the resource allocation schedule to a second entity;
causing generation of respective scheduling data for the first entity and the second entity, wherein the scheduling data corresponds to the performance times;
providing the respective scheduling data to the first and second entities, wherein providing the respective scheduling data comprises causing display of appointment information;
receiving, from a second user or system, a health update about the subject that causes an adjustment of priority of one or more tasks of the set of tasks;
in accordance with receiving the health update, generating an updated resource allocation schedule by updating one or more parameters of the resource allocation schedule according to the adjustment of priority, including generating updated respective scheduling data, wherein the updated respective scheduling data corresponds to different performances times than the respective scheduling data;
providing an indication of the updated resource allocation schedule to the first user; and
providing the updated respective scheduling data to the first and second entities and to the first user.

21. The method of claim 1, wherein the at least a subset of the information about the subject, the at least a subset of the first set of parameters for the first subset of core tasks, and the additional information obtained from one or more databases are provided to the AI engine as a vector of inputs.

22. The method of claim 1, wherein the first set of parameters further indicates a set of two or more potential owners for a particular task of the set of tasks, and wherein the one or more ownership parameters of the second set of parameters indicate a particular owner from the set of two or more potential owners.

23. The method of claim 1, wherein providing the respective scheduling data to the first and second entities comprises causing a set of calendar entries to be generated from the first and second entities.

24. The method of claim 1, wherein generating the updated resource allocation schedule comprises identifying, via the AI engine, one or more additional candidate tasks for the resource allocation schedule based on the health update about the subject.

25. The method of claim 1, wherein providing the updated respective scheduling data to the first and second entities and to the first user comprises causing display of respective updated appointment information for the first and second entities.
